(19) United States
(12) Patent Application Publication
(10) Pub. No.: US 2019/0076265 A1
(43) Pub. Date: Mar. 14, 2019

(54) One Lumbar Zero-profile Fusion System

(71) Applicant: Beijing Fule Science & Technology Development Co., Ltd., Beijing (CN)

(72) Inventors: Lubomír Subert, Prague (CZ); Yaling WANG, Beijing (CN); Guoping FAN, Beijing (CN); Xiyi HUANG, Beijing (CN); Guangpeng CAI, Beijing (CN); Wanyu QIU, Beijing (CN)

(21) Appl. No.: 16/083,877

(22) PCT Filed: Oct. 13, 2016
(86) PCT No.: PCT/CN2016/102013

§ 371 Date: Sep. 10, 2018

(30) Foreign Application Priority Data
Aug. 30, 2016 (CN) 2016107688717
Aug. 30, 2016 (CN) 2016209934764

(51) Int. Cl. A61F 2/44

(52) U.S. Cl. CPC A61F 2/4455; A61F 2/30767

(57) ABSTRACT

The invention discloses a one lumbar zero-profile fusion system, including a holder, opener and nailing machine. The fusion system contains a fuser body, front support block, rear support block and adjustable lever; the fuser body covers the upper fuser body I as well as lower fuser body II, matching with the upper fuser body I; both ends of the fuser body connect to the front support block and rear support block, both of which connect to an adjustable lever; the adjustable lever goes through the said fuser body; rear support block and fuser body are connected with a fixed plate; the left and right side of the fixed plate are equipped with oblong holes which are connected to the fuser body by screw axle; and, the holder is equipped with long circular projection matching with the said oblong hole.

### TECHNICAL FIELD OF THE INVENTION

The invention relates to the medical device technology field, specifically one kind of lumbar zero-profile fusion system.

### BACKGROUND OF THE INVENTION

When lesion or necrosis affects the intervertebral disc of the spine, it's necessary to undergo interbody fusion surgery. The traditional surgery procedure is to remove the intervertebral disc suffering from the lesion and implanting the interbody fusion cage matching with the shape of the intervertebral disc in the place where intervertebral disc is removed. Interbody fuser structures currently used include interbody fusion cage noumenon, having the disadvantages of whose a low holding force between interbody fusion cage and nearby spinal block, as well as bad stability. It is therefore necessary to place one piece of steel sheet on one side of the interbody fusion cage, and both ends of such steel sheet are fixed to vertebral body next to interbody fusion cage with bolts. As a result, the surgery becomes complicated, especially when the intervertebral disc of human cervical vertebra needs the above interbody fusion cage; placing the steel sheet higher than the cervical vertebra surface on the anterior cervical spine goes against blood supply, resulting in strong foreign body sensation after the surgery, and increases the occurrence rate of difficulty in swallowing after the surgery because there are esophagus and weasand.

During fusion surgery, in general, cooperation among several apparatuses is necessary. For example, the holder holds and conveys the fuser to a corresponding place, the opener throws the fuser into the corresponding diseased region and then the fuser is fixed by the nailing machine.

Chinese patent application number of 201110409848.6 discloses one interbody fusion cage, including fusion body and pin. The fusion body is equipped with pin holes and channel for filling pedicle, and the pin is installed in the pin hole for the purpose of connecting and combining more than two fusion bodies. However, two fusion bodies of the above patent are connected by a pin; as a result, the a loose connection might occur. When the pin disconnects from the fuser body, it's easy that the fusion process is influenced and even the normal proceeding of fusion will be influenced. Additionally, the structure of the above fuser cannot get close to the vertebral plate, so it's easy to cause injury in the surgery process.

Furthermore, existing auxiliary instruments include a holder, opener, nailing machine, etc., whose disadvantages are inconvenient operation, complicated structure and being inconvenient for interbody fusion going with a swing.

### SUMMARY OF THE INVENTION

The invention is aimed at providing one lumbar zero-profile fusion system in order to overcome the above shortcomings of current technologies.

In order to achieve the above technical object, the technical solution of the present invention is achieved by the following:

The lumbar zero-profile fusion system includes a holder, opener and nailing machine. The said fusion system contains a fuser body, front support block, rear support block and adjustable lever. The fuser body covers an upper fuser body I as well as a lower fuser body II, matching with upper fuser body I. Both ends of the said fuser body connect to a front support block and rear support block, both of which connect to adjustable lever. The adjustable lever goes through the said fuser body. The rear support block and fuser body are connected with a fixed plate. The left and right side of the said fixed plate are equipped with the said oblong holes which are connected to fuser body by screw axle. The said holder is equipped with long circular projection, matching with the said oblong hole. Internal moving parts of the holder are connected to the opener, matching and connecting with the said adjustable lever. The fixed plate is equipped with four cylindrical holes with ball sockets and is connected to the lock screw through the said cylindrical hole.

Further, the said fixed plate is arc-like, and there is a rectangular groove installed in the middle of the said fixed plate, which is used to install the rear support block. Both sides of the said rectangular groove are equipped with stripped projection and the rear support block is equipped with a rectangular gap matching with the said stripped projection.

Additionally, there are press stud holes between two cylindrical holes in the left side of the said fixed plate as well as between two cylindrical holes in the right side of the said fixed plate. The fixed plate is connected with press stud through the said press stud hole. There are two arc gap on the nail head of the said press stud.

Further, the said fuser body is equipped with cylindrical holes which the said adjustable lever goes through, and the said adjustable lever is of step columnar structure. Both ends of the said adjustable lever are equipped with threads. The said front support block and rear support block are equipped with threaded holes matching with the said thread.

Moreover, one side of the said upper fuser body I is equipped with projection a block I and groove I. Another side of the said upper fuser body I is equipped with groove II and projection block II, corresponding to the projection block I and groove I. The downside of the said upper fuser body I upper groove I is designed with cylindrical projection. The downside of the said upper fuser body I is designed with the long cylindrical holes corresponding to the said cylindrical projection.

Additionally, two ends of the said fuser body are designed to dovetail the groove. The front support block and the rear support block are designed with the dovetail projection block that matches with the said dovetail groove. The outside of the said fuser body includes a detent.

Further, the holder includes the holding rod. The head of the holding rod includes the long circular projection that matches with the said oblong holes. The head of the said holding rod is designed with long slotting. The external sleeve of the holding rod is designed with the outer jacket, on the head of which jacket claw is designed to match with the head of the holding rod.

Further, the tail of the said holding rod is designed with four U-shaped grooves, and a drop-proof boss is set at the tail end of the said holding rod. The said holding rod is connected to the knobs via the U-shaped groove. Threaded holes are set on the knob. External thread is set on the outer jacket. The knobs are connected to the threaded holes via the external thread.

Furthermore, the said outer jacket is fixed to the connection rod, which is designed with silicone handle fixed by pins.

Moreover, the opener includes an opener rod and a horizontal handle that is fixed to the opener rod. Holes for threading the said opener rod are equipped on the said knob and holding rod. The left side of the said opener rod is designed with hexagonal protruding parts. Hexagonal holes are equipped on the end that connects the adjustable lever and the rear support block. The opener rod is inserted into the hexagonal hole via the hexagonal protruding parts to connect with the adjustable lever.

If the above technical scheme is adopted, the present invention will have the following beneficial results: The fuser body of the present invention is composed of two parts, and the height is adjustable to abutt the neural plate. The design meets the requirement of physiological curvature, so it is convenient for operative procedures and beneficial for reducing damages and improving the supporting stability of fuser. The fixed plate and interbody fusion cage of the present invention are pre-assembled. After they are placed in, the fixed plate will fit automatically, which is convenient for the operation procedures and saves the trouble of combing it with spinal nail bat and other products. Screw down the lock screw tightly to fix the fuser body to the intervertebral space. Combine the press stud 7 and the fixed plate 4 to avoid the dropping of lock screws and enhance the use safety. The holder of the present invention has a simple structure, which can hold the fuser body to the corresponding position. Run the opener throughout the holder inside to match and connect with the adjustable lever of the fuser. In this case, rotate the opener to drive the rotation of adjustable lever, and further open the fuser to realize the effect of opening the interbody.

### BRIEF DESCRIPTION OF THE DRAWINGS

To better describe the cases adopting this invention or the technical schemes of current technologies, a brief introduction of the attached figures to be used in the descriptions of application cases or current technologies is presented. Obviously, the attached figures described below are only several application cases of this invention. For common technicians in this field, they can obtain other attached figures based on these without making additional creative endeavors.

FIG. 1 is the front view of the fuser used in the present invention.

FIG. 2 is the side view of the fuser used in the present invention.

FIG. 3 is the front view of the upper fuser body I used in the present invention.

FIG. 4 is the view of FIG. 3 in direction of B.

FIG. 5 is the section view of FIG. 4 in the direction of E-E.

FIG. 6 is the front view of the front support block used in the present invention.

FIG. 7 is the side view of the front support block used in the present invention.

FIG. 8 is the vertical view of the front support block used in the present invention.

FIG. 9 is the front view of rear support block used in the present invention.

FIG. 10 is the axonometric drawing of rear support block used in the present invention.

FIG. 11 is the axonometric drawing of fixed plate used in the present invention.

FIG. 12 is the structure diagram of holder used in the present invention.

FIG. 13 is the structure diagram of opener used in the present invention.

The picture: 1. Front support block; 2. Fuser body; 3. Screw axle; 4. Fixed plate; 5. Rear support block; 6. Adjustable lever; 7. Press stud; 8. Lock screw 9. Lower fuser body 2; 10. Upper fuser body 1; 11. Oblong hole; 12. Cylindrical hole; 13. Stripped projection; 14. Rectangular gap; 15. Press stud hole; 16. Curved notch; 17. Projection block I; 18. Groove I; 19. Groove II; 20. Projection block II; 21. Cylindrical projection; 22. Long cylindrical hole; 23. Dentation; 24. Holding rod; 25. Outer jacket; 26. Long circular projection; 27. Long slotting; 28. Jacket claw; 29. U-shaped groove; 30. Drop-proof boss; 31. Knob; 32. Connecting rod; 33. Pin; 34. Silicone handle; 35. Opener rod; 36. Horizontal handle; 37. Hexagonal protruding part.

### DETAILED DESCRIPTION OF THE INVENTION

A clear and complete description of the technical schemes in application cases of this invention is performed while combining the attached figures of such cases. Obviously, only some application cases of this invention (instead of all the cases) are described here. Based on the examples in the present invention, all other examples obtained by the common technicians in the field shall be in the scope of protection of the present invention.

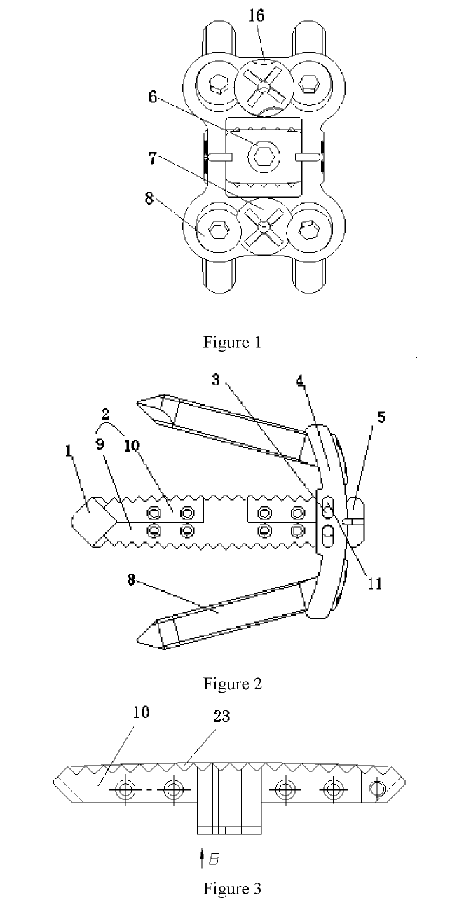

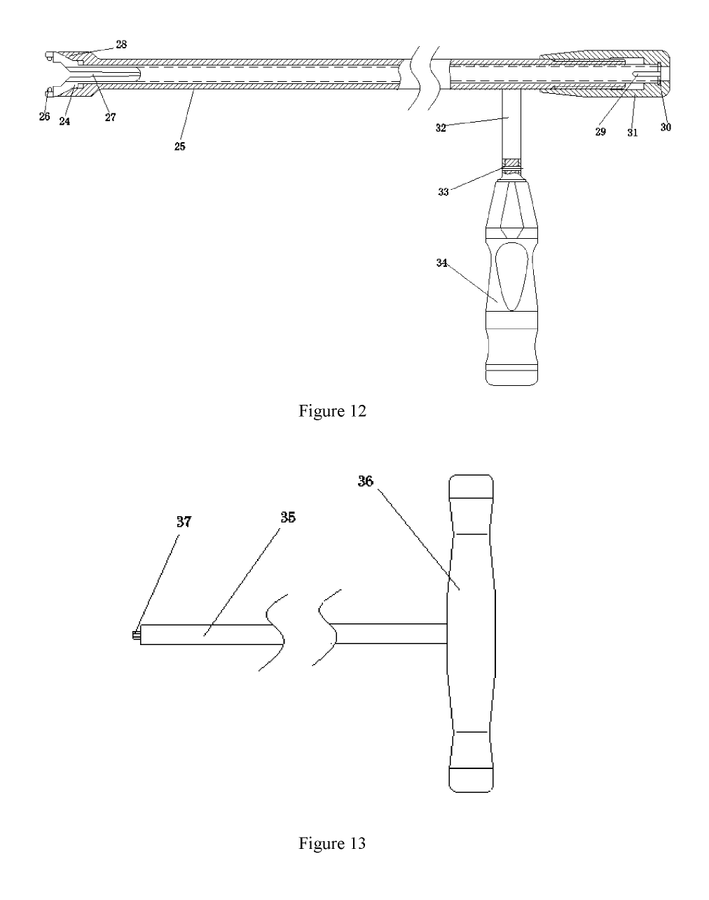

As shown in FIG. 1-2 and FIG. 12-13, the lumbar zero-profile fusion system described in the example of the present invention is composed of a fuser, holder, opener and nailing machine. The said fuser includes the fuser body 2, front support block 1, rear support block 5 and adjustable lever 6. The said fuser body 2 contains upper fuser body I 10 and lower fuser body II 9, matching with the upper Fuser body I 10. Both ends of the said fuser body 2 are connected to front support block 1 and rear support block 5, respectively, both of which connect to adjustable lever 6. The said adjustable lever 6 goes through the said fuser body 2; the said rear support block 5 is connected to fixed plate 4. Left and right sides of the said fixed plate 4 are equipped with oblong hole 11 which connects to fuser body 2 through screw axle 3. The said holder is equipped with long circular projection 26 matching with the said oblong hole 11. Internal moving parts of the said holder are connected to the opener. The opener is matched and connected with the adjustable lever 6. The fixed plate 4 is equipped with four cylindrical holes 12 with a ball socket. The fixed plate 4 is connected with lock screw 8 through the said cylindrical hole 12.

Wherein, as shown in FIG. 12, the said holder 24 includes the holding rod. The head of the holding rod 24 is designed with the long circular projection 26 that matches with the oblong holes 11. The head of the holding rod 24 is designed with long slotting 27. The external sleeve of the holding rod 24 is designed with the outer jacket 25, on the head of which jacket claw 28 is designed to match with the head of the holding rod 24. There is a long circular projection 26 that matches with oblong hole 11 on both sides of fuser fixed plate 4 on the head of holding rod 24. It is used to hold the fuser. The head of holding rod 24 is designed with a long slotting 27, which can close and open under the action of outer jacket 25 to reach the effect of tightening and loosening the fuser for the convenience of operative procedures. The outer jacket 25 is hollow inside, in which the holding rod 24 can be inserted. Thread is designed at the tail end, and the left head is designed with a pair of jacket claws 28, which are used together with the head of holding rod 24. The holding rod 24 will clamp the fuser when the outer jacket 25 is pushed to the left, while the holding rod will loosen the fuser when the outer jacket is rotated to the right.

As shown in the FIG. 13, the opener includes opener rod 35 and horizontal handle 36 that is fixed to the opener rod 35. Holes for threading the said opener rod 35 are equipped on the said knob 31 and holding rod 24. The left side of the opener rod 35 is designed with hexagonal protruding parts 37. Hexagonal holes are equipped on the end that connects the said adjustable lever 6 and the rear support block 5. The said opener rod is inserted into the said hexagonal hole via the hexagonal protruding parts 37 to connect with the said adjustable lever. Through-holes on holding rod 24 and knob allow the opener to run through. The left end of opener rod 35 is designed with the hexagonal protruding part 37, which can be used together with hexagonal holes to open the fuser.

The said nailing machine is composed of a cross wrench lever, inner jacket, outer jacket, pins, lining and small vertical handle. The small vertical handle is composed of a bakelite rod, inner core, pins and rotation blocks. The left end of the cross wrench is in a shape of the cross, which is used to rotate the screws. It is close to the step shaft in the rear. The close contacting part of step shaft and inner jacket ensures the coaxiality of the cross wrench lever and inner jacket. The untouched part of step shaft and inner jacket make the cross wrench lever easier to be inserted in the inner jacket. The external left side of the inner jacket is composed of a shaft with conicity connected to the circular shaft and a hole, which is ladder-like inside. There are symmetrical openings on both sides, which are used to extend the left-end shaft for the convenience of installation and use. The right end is a grooved shaft. The outer jacket inside is a coned hole connecting with the ladder-like shaft hole, and the external right end is designed with four grooves, and the disc-like design on the right end is used to move the handle when adjusting the assemblies. There is hole matching with the cross wrench lever inside the left side of lining cylindrical shaft, and a pin hole is designed at the right to fix the small vertical handle. The main function of the nailing machine is to implant the lock screw 8 in the neural plate to fix the fuser and avoid movement.

In one specific case, the tail of the said holding rod 24 is designed with four U-shaped grooves 29, and a drop-proof boss 30 is set at the tail end of the said holding rod 24. The said holding rod 24 is connected to the knobs 31 via the said U-shaped groove 29. Threaded holes are set on the said knob 31. External thread is set on the outer jacket 25. The said knobs 31 are connected to the threaded holes via the external thread 25. The tail end of holding rod is designed with four U-shaped grooves 29 and a drop-proof boss 30. The U-shaped groove 29 is convenient to be compressed and installed into the knob 31, and the drop-proof boss 30 can prevent the holding rod 24 from dropping off the knob 31. A section of threaded holes are set in the knob 31 to cooperate with the threaded part at the tail end of the outer jacket. Step holes are designed in the knob 31, and the holding rod 24 and drop-proof boss 30 can be clipped into the step hole. When the knob 31, outer jacket 25 and holding rod 24 are assembled, the knob 31 can be rotated to remove the holding rod 24 towards the left or the right against the outer jacket 25 by connecting to the thread of outer jacket 25.

In another preferred embodiment, the connection rod 32 is fixed to the said outer jacket 25, and the silicone handle 34 is fixed to the said connection rod via the pin 33. The said silicone handle 34 is used to assist the operation by coordinating the knob to tighten or loosen the holding rod 24 and further to tighten or loosen the fuser.

In another embodiment, as shown in FIG. 11, the fixed plate 4 is arc-like, and there is a rectangular groove installed in the middle of the said fixed plate 4, which is used to install the rear support block 5. Both sides of the rectangular groove are equipped with stripped projection 13 and the said rear support block 5 is equipped with rectangular gap 14 matching with the said stripped projection. The fixed plate is designed with a step-like cylindrical hole which has an angle of −15° with the horizontal direction. It is used to screw in the lock screws. The stripped projection on the fixed plate is clipped into the rectangular gap of the rear support block to limit its movement upwards and downward. A rectangular hole is designed at the center, which can be clipped into the boss of fuser body.

In another embodiment, there are press stud holes 15 between two cylindrical holes in the left side of the said fixed plate 4, as well as between two cylindrical holes 12 in the right side of the said fixed plate. The said fixed plate 4 is connected with press stud 17 through the said press stud hole 15. There are two arc gaps 16 on the nail head of the said press stud 7. An arc gap is designed on the press stud, and the stud body is designed with thread. Implant the press stud in the threaded hole with a circular groove on the fixed plate, and screw the nail head until the side without the gap is screwed down to the step-like cylindrical hole to prevent the lock screw from dropping off.

In another preferred embodiment, the said fuser body 2 is equipped with cylindrical holes, which the said adjustable lever 6 goes through, and the said adjustable lever 6 is of step columnar structure. Both ends of the said adjustable lever 6 are equipped with threads. The said front support block 1 and rear support block 2 are equipped with threaded holes matching with the said thread.

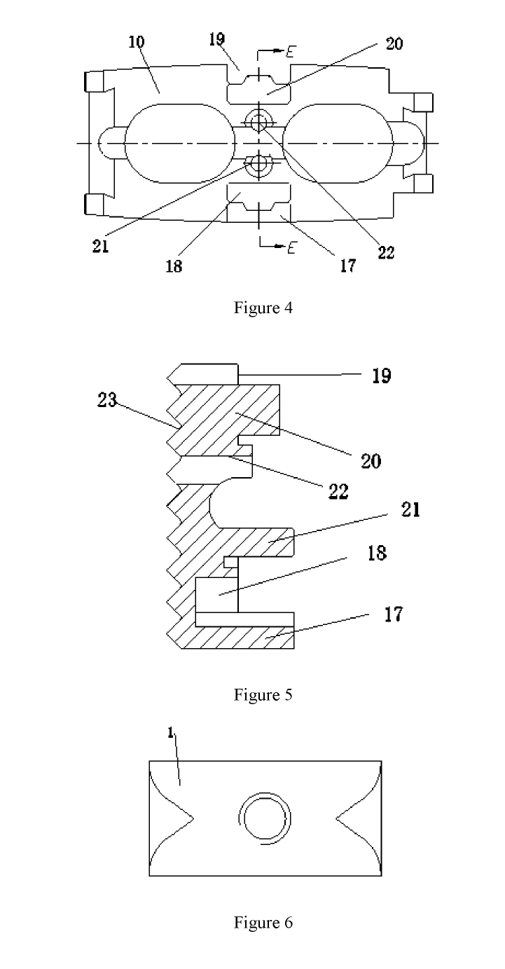

In another preferred embodiment, as shown in FIGS. 3-5, one side of the said upper fuser body I 10 is designed with projection block I 17 and groove I 18, and the other side is designed with the groove II 19 and the projection block II 20 corresponding to the said projection block I 17 and the groove I 18. The downside of the groove I 18 on the said upper fuser body I 10 is equipped with cylindrical projection 21, and the downside of the said upper fuser body I 10 is equipped with a long cylindrical hole 22 corresponding to the said cylindrical projection 21. The lower fuser body II 9 has the same structure with the upper fuser body I 10. In installation, the upper fuser body I is connected to the lower fuser body II by the matching of convex and concave, wherein the groove is connected to the projection block and the cylindrical projection is connected to the long cylindrical hole (for example, the boss of one fuser body is matched with the groove of the other boss, similar to the cylinder and the cylindrical hole.). Grooves are designed on the projection and the cylindrical projection to limit the two fuser bodies, such that they can only be removed upwards and downwards. The fuser body is designed with an arc groove, which can accommodate the reducing adjustable lever; Bone crushers can be implanted into the designed bone hole to accelerate the treatment process.

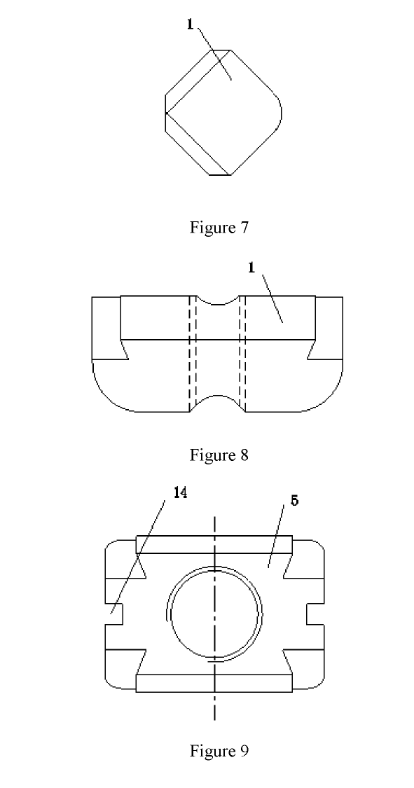

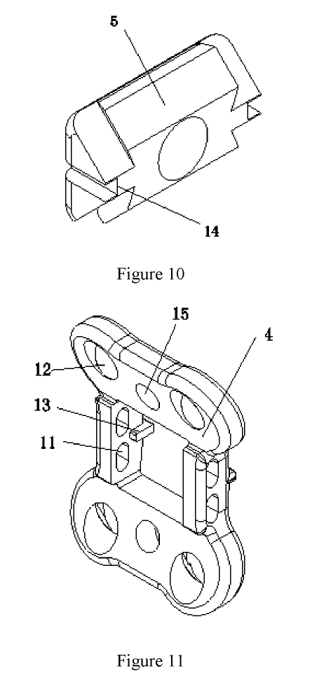

In another preferred embodiment, as shown in FIGS. 6-10, dovetail grooves are designed on both sides of the said fuser body, and the dovetail bosses are designed for the said front support block 1 and rear support block 5. Threaded holes are set on the dovetail boss, and the adjustable lever run through the said threaded hole to connect the front support block and the rear support block.

In another embodiment, the said upper fuser body I 10 and the said lower fuser body II 9 are designed with dentents 23 outside. The dentents 23 on the fuser body can significantly improve the anti-sliding force in all directions to avoid the fuser movement.

To better understand the present invention, the application steps of the present invention will be described as follows according to the operating steps of the operation: 1. During the operation, excise the intervertebral disc suffering from lesion, repair and maintain the neural plate to make a good preparation of fuser implant at first. 2. The holding rod head of holder can be matched and connected to the oblong holes on both sides of fuser fixed plate, rotate the outer jacket to the left to tighten the lumbar zero-profile fuser, and then implant the fuser into the center of two neural plates. At the moment, the fuser is not opened. After implanting the fuser, run the opener throughout the holder inside to match and connect with the adjustable lever of fuser. In this case, rotate the opener to drive the rotation of adjustable lever, and further open the fuser to realize the effect of opening the interbody; 3. Implant 4 lock screws in the neural plate by the nailing machine, fix the fuser to prevent the displacement. 4. Screw down the press stud to prevent the lock screws from dropping off and make the implanted fuser more stable.

The above description describes only the optimal examples of the present information, but is not intended to limit the present information. Any modification, equivalent replacement, improvement, etc. within the spirit and principle of the present information are all covered in the scope of protection of the present invention.

1. A one lumbar zero-profile fusion system comprising a fuser, holder, opener and nailing machine, wherein the fuser covers fuser body, front support block, rear support block and adjustable lever, wherein the said fuser body contains an upper fuser body I and lower fuser body II matching with upper fuser body I, wherein both ends of the fuser body are connected to the front support block and rear support block, respectively, both of which connect to an adjustable lever, wherein the said adjustable lever goes through the said fuser body, wherein the rear support block and fuser body are connected to a fixed plate, wherein the left and right sides of the fixed plate are equipped with an oblong hole which connects to the fuser body through screw axle, wherein the said holder is equipped with long circular projection matching with the said oblong hole wherein internal moving parts of the said holder are connected to an opener wherein the said opener is matched and connected with the said adjustable lever, wherein the said fixed plate is equipped with four cylindrical holes with a ball socket, wherein the said fixed plate is connected with a lock screw through the said cylindrical hole.

2. The one lumbar zero-profile fusion system of claim 1, wherein the said fixed plate is arc-like and there is a rectangular groove for installing the rear support block in the middle of the said fixed plate, wherein both sides of the said rectangular groove are equipped with stripped projection and the said rear support block is equipped with rectangular gap matching with the said stripped projection.

3. The one lumbar zero-profile fusion system of claim 1, wherein there are press-stud holes between two cylindrical holes in the left side of the said fixed plate, as well as between two cylindrical holes in the right side of the said fixed plate, wherein the said fixed plate is connected with press button stud through the said press stud hole and there are two arc gaps on the nail head of the said press button stud.

4. The one lumbar zero-profile fusion system of claim 1, wherein the said fuser body is equipped with cylindrical holes which the said adjustable lever shall go through, the said adjustable lever is of step-columnar structure, wherein both ends of the said adjustable lever are equipped with threads and the said front support block and rear support block are equipped with threaded holes matching with the said threads.

5. The one lumbar zero-profile fusion system of claim 1, wherein by one side of the said fuser body I is equipped with projection block I and a groove, wherein another side of the said upper fuser body I is equipped with groove II and projection block II corresponding to the said projection block I and groove I, wherein the downside of the groove I on the said upper fuser body I is equipped with a cylindrical projection, and the downside of the said upper fuser body I is equipped with a long cylindrical hole corresponding to the said cylindrical projection.

6. The one lumbar zero-profile fusion system stated of claim 5, wherein both ends of the said fuser body are equipped with a dovetail groove, wherein the said front support block and rear support block are equipped with dovetail projection block matching with the dovetail groove and the outer side of the said fuser body is equipped with a detent.

7. The one lumbar zero-profile fusion system of claim 1, wherein the holder includes a holding rod, the head of the said holding rod is equipped with long circular projection matching with the said oblong hole, long slotting and outer jacket, and the head of the said outer jacket being equipped with jacket claw matching with the head of holding rod.

8. The one lumbar zero-profile fusion system of claim 7, wherein the tail of the said holding rod is equipped with four U-shape grooves and the drop-proof boss, wherein the holding rod is connected to rotary knob through the said U-shaped groove, wherein the said knob is equipped with a threaded hole, wherein the said outer jacket is equipped with external thread and the said knob and outer jacket is matched and connected with each other through external thread and threaded hole.

9. The one lumbar zero-profile fusion system of claim 8, wherein the said outer jacket is firmly connected with connecting rod and the said connecting rod is firmly connected to a silicone handle through pins.

10. The one lumbar zero-profile fusion system of claim 8, wherein the said opener contains opener rod and transverse handle is firmly connected with the opener rod the said knob and holding rod is equipped with holes which the said opener rod goes through, wherein the left side of the said opener rod is equipped with hexagonal projection part, wherein one end of the said adjustable lever connecting to rear support block is equipped with a hexagonal hole inside, and the said opener rod entering the said hexagonal hole and the said adjustable lever flexible connection through the said hexagonal projection part.

11. The one lumbar zero-profile fusion system of claim 9, wherein the said opener contains opener rod and transverse handle is firmly connected with the opener rod, the said knob and holding rod is equipped with holes which the said opener rod goes through, wherein the left side of the said opener rod is equipped with hexagonal projection part, wherein one end of the said adjustable lever connecting to rear support block is equipped with a hexagonal hole inside, and the said opener rod entering the said hexagonal hole and the said adjustable lever flexible connection through the said hexagonal projection part.
